(19) United States
(12) Patent Application Publication
(10) Pub. No.: US 2005/0032117 A1
(43) Pub. Date: Feb. 10, 2005

(54) Method for assessment of cystic lung fibrosis

(75) Inventors: Richard B. Moss, Palo Alto, CA (US); Akitoshi Ishizaka, Shinagawa-ku (JP); Teruo Kirikae, Shinjuku-ku (JP)

Correspondence Address: SUGHRUE MION, PLLC, 2100 PENNSYLVANIA AVENUE, N.W., SUITE 800, WASHINGTON, DC, 20037, US

(73) Assignee: SEIKAGAKU CORPORATION

(21) Appl. No.: 10/777,683

(22) Filed: Feb. 13, 2004

Related U.S. Application Data
(60) Provisional application No. 60/447,310, filed on Feb. 14, 2003.

(51) Int. Cl. G01N 33/53

(52) U.S. Cl. 435/7.1

(57) ABSTRACT

The invention provides a method for assessment of cystic lung fibrosis (CF) in terms of severity, acuteness, degree of progress, etc. of CF., with high accuracy, high sensitivity, convenience, rapidity, and low cost. The method includes the steps of measuring the level of CAP 18 in a biological sample, and correlating the measurement with CF.

### BACKGROUND OF THE INVENTION

1. Field of the Invention

The present invention relates to a method for assessment of cystic lung fibrosis and to a kit for assessment of cystic lung fibrosis.

2. Related Art

The following abbreviations are employed in the present specification.
BALF: Bronchoalveolar lavage fluid
BSA: Bovine serum albumin
CF: Cystic lung fibrosis
FBS: Fetal bovine serum
HRP: Horseradish peroxidase
PBS: Phosphate-buffered saline
TMB: 3,3′,5,5′-Tetramethylbenzidine

CF collectively refers to types of lung fibrosis that form cysts, and forms a class of hereditary diseases (autosomal recessive gene; abnormality in the CFTR gene). Patients suffering from CF have disturbed exocrine glands in the whole body (lungs, pancreas, digestive organs, sweat glands, etc.), and tend to experience complication with intractable infections by, for example, pseudomonas aeruginosa or staphylococci.

CAP 18 (cationic antimicrobial protein of 18 kDa) is a basic antimicrobial protein identified in human or rabbit granulocytes, and is known to exhibit a broad antimicrobial activity against gram negative bacteria and gram positive bacteria. The entire amino acid sequence of human CAP 18 is described in, for example, Japanese Kohyo (PCT) Patent Publication No. 8-504085.

Hitherto, measurement of CAP 18 has not been known to be a means for assessing CF.

Conventionally known diagnosis methods for CF have typically been established on the basis of either genotypic features of CF (CFTR mutations) or phenotypic features of CF (sweat electrolyte value). However, there remains a need for a more accurate, highly sensitive, more convenient, quicker, and inexpensive assessment method for facilitated control of CF (assessment of severity or acuteness of CF, assessment of the degree of progress of CF, etc.).

The present inventors have performed extensive studies with an aim toward attaining the above goal, and have found that measurement of CAP 18 in a biological sample achieves assessment of CF with high accuracy, high sensitivity, convenience, rapidity, and low cost, thus leading to completion of the invention.

### SUMMARY OF THE INVENTION

The present invention provides a method for assessment of CF, comprising measuring the level of CAP 18 in a biological sample, and correlating the measurement with CF (hereinafter referred to as “the present method”).

Preferably, the present method includes the following steps (1) to (3):

(1) a step of measuring the level of CAP 18 contained in a biological sample collected from an individual;

(2) a step of comparing the CAP 18 level determined in step (1) with the level of CAP 18 in a control sample; and

(3) a step of correlating the result from step (2) with CF.

The “biological sample” used in the method of the present invention is preferably an expectoration or BALF.

In the present invention, the “CAP 18 level” is preferably measured through antigen-antibody reaction, which is preferably carried out by use of “an antibody capable of binding to a peptide having an amino acid sequence of SEQ ID NO: 1.”

Preferably, the measurement making use of an antigen-antibody reaction employs a solid phase, and more preferably includes at least the following steps (a) to (c):

(a) a step of bringing a sample into contact with a solid phase, to thereby immobilize onto the solid phase CAP 18 contained in the sample;

(b) a step of causing the immobilized CAP 18 obtained in step (a) to be bound to “an antibody capable of binding to a peptide having an amino acid sequence of SEQ ID NO: 1,” to thereby form a complex of the two components; and

(c) a step of detecting the complex formed in step (b).

The method including the above steps (a) to (c) is hereafter referred to as Method 1.

Alternatively, measurement making use of an antigen-antibody reaction employing a solid phase is preferably carried out through a method including at least the following steps (a)′ and (b)′:

(a)′: a step of bringing into mutual contact the following three components; i.e., a solid phase to which a first antibody capable of binding to a peptide having an amino acid sequence of SEQ ID NO: 1 has been immobilized, a sample, and a second antibody capable of binding to a peptide having an amino acid sequence of SEQ ID NO: 1, to thereby form a sandwich-like complex formed of “first antibody immobilized onto a solid phase—CAP 18—second antibody”; and

(b)′ a step of detecting the sandwich-like complex formed in step (a)′.

The method including the above steps (a)′ and (b)′ is hereafter referred to as Method 2.

Preferably, the “assessment” according to the present invention is selected from the group consisting of diagnosis; determination of the presence or absence of risk, or assessment of the level of the risk; assessment of severity and/or acuteness; and assessment regarding progress of disease.

The present invention also provides a kit for assessment of CF, containing at least the following components (A) and (B):

(A) a solid phase, and

(B) an antibody capable of binding to a peptide having an amino acid sequence of SEQ ID NO: 1.

The kit including the above components (A) and (B) is hereafter referred to as Kit 1.

The present invention also provides a kit for assessment of CF, containing at least the following components (A)′ and (B)′:

(A)′: a solid phase to which a first antibody capable of binding to a peptide having an amino acid sequence of SEQ ID NO: 1 has been immobilized, and

(B)′: a second antibody capable of binding to a peptide having an amino acid sequence of SEQ ID NO: 1.

The kit including the above components (A)′ and (B)′ is hereafter referred to as Kit 2.

Hereafter, the Kits 1 and 2 are collectively referred to simply as the present Kit.

Various other objects, features and many of the attendant advantages of the present invention will be readily appreciated as the same becomes better understood by reference to the following detailed description of the preferred embodiments.

### DETAILED DESCRIPTION OF THE PREFERRED EMBODIMENTS

Modes of the present invention will next be described.

<1> The Present Method

The present method is directed to assessment of CF including at least the steps of measuring the CAP 18 level in a biological sample and correlating the results of measurement with CF.

Preferably, the present method includes the following steps (1) to (3):

(1) a step of measuring the level of CAP 18 contained in a biological sample collected from an individual;

(2) a step of comparing the CAP 18 level determined in step (1) with the level of CAP 18 contained in a control sample; and

(3) a step of correlating the result from step (2) with CF.

According to the present invention, no particular limitations are imposed on the biological sample, so long as the sample is derived from a living organism and the amount of CAP 18 contained in the sample varies in relation to CF. That is, the expression “sample derived from a living organism” not only refers to a sample directly extracted from a living organism carrying the sample, but encompasses any type of excretion which is collected through administration of a liquid or a similar substance to a living organism for causing excretion, or a dilution of such an excretion after collection (note that, in either case, the sample will be diluted with the same liquid or with the aforementioned similar substance). Moreover, a supernatant, a precipitate obtained through centrifugation of the collected sample, and a similar substance should also be considered a “sample derived from a living organism.”

Preferably, the above-described “biological sample” is an expectoration or BALF.

No particular limitations are imposed on the individual from which a biological sample is collected, so long as the individual is an animal individual which may possibly suffer from CF. A preferred example of such an individual is a mammalian, with a human being more preferred.

As mentioned above, CAP 18 is a protein whose amino acid sequence has already been known. For the purpose of illustration, the entire amino acid sequence of human CAP 18 is appended hereto (SEQ ID NO: 4).

A naturally occurring protein may undergo substitution, deletion, insertion, transposition, or other alterations in its amino acid sequence, as a result of mutation or polymorphism of the DNA encoding the protein or intravital modification occurring after the protein has been biosynthesized. Nevertheless, some proteins are known to exhibit physiological or biological activities substantially equivalent to those of their corresponding polypeptides having no mutations. Therefore, the expression “CAP 18,” which is the object of assessment of the present invention, encompasses proteins having slight structural differences from native (non-mutated) CAP 18 but exhibiting no significant differences in terms of intravital function, behavior, etc.

As used herein, the term “level” (or “amount”) may be either qualitative (presence or absence of CAP 18) or quantitative. In the latter case, the “level” (or “amount”) may be represented by either a specific numerical value or by a degree or extent (for example, “high” or “low”). Moreover, the “level” (or “amount”) may be represented by any of concentration, weight, number of molecules (or mol number), and similar indices.

When CAP 18 is measured by means of any of absorbance, counts of radioactivity, fluorescence intensity, luminance intensity, etc., the measurement may be directly employed as an index of the “level” (or “amount”) of CAP 18. Alternatively, the data may be processed by use of a previously prepared calibration curve or a correlation formula, to thereby calculate the concentration, weight, number of molecules (or mol number), etc. of CAP 18.

The level of CAP 18 may be determined by any method capable of distinguishing CAP 18 from other components and allowing detection and measurement of CAP 18 contained in a biological sample. One such method is a method employing antigen-antibody reaction.

That is, CAP 18 present in a biological sample can be measured through use of an antigen-antibody reaction employing an “antibody capable of achieving specific binding to CAP 18.” As used herein, no particular limitations are imposed on the “antibody capable of achieving specific binding to CAP 18,” so long as the antibody can be bound to CAP 18 in a selective manner. Preferably, the antibody capable of achieving specific binding to CAP 18 is an antibody capable of binding to a peptide having an amino acid sequence of SEQ ID NO: 1. Preferably, the “antibody capable of binding to a peptide having an amino acid sequence of SEQ ID NO: 1” is either an “antibody capable of binding to a peptide having an amino acid sequence of SEQ ID NO: 2” or an “antibody capable of binding to a peptide having an amino acid sequence of SEQ ID NO: 3.”

These antibodies may be polyclonal or monoclonal, but in general, from the viewpoints of specificity, homogeneity, reproducibility, massive and long-run productivity, etc., monoclonal antibodies are preferred.

These antibodies may be Fab-containing fragments treated with a protease which does not digest antigen-binding sites (Fab) (examples of such a protease include plasmin, pepsin, and papain). Examples of Fab-containing fragments of an antibody include Fab, Fabc, and (Fab′)₂. Thus, in relation to the present invention, the term “antibody” should be construed broadly.

The “antibody capable of binding to a peptide having an amino acid sequence of SEQ ID NO: 1” can be obtained by a conventional method for producing an antibody, through use, as an antigen, of a peptide having an amino acid sequence of SEQ ID NO: 1 (i.e., the SEQ ID NO: 1 peptide per se) or a partial peptide thereof (hereinafter these peptides may be collectively referred to as antigen peptides).

Examples of the partial peptides include a peptide having an amino acid sequence of SEQ ID NO: 2, and a peptide having an amino acid sequence of SEQ ID NO: 3.

Such an antigen peptide can be produced by a conventionally known chemical synthesis method—such as the liquid phase synthesis method or the solid phase synthesis method—on the basis of the sequence thereof. Alternatively, a polynucleotide (DNA or RNA) corresponding to the amino acid sequence of the antigen peptide is produced, and the obtained polynucleotide is subjected to genetic engineering.

The amino acid sequence of the thus-produced antigen peptide is determined through a conventionally known amino acid sequencing method, such as the Edman degradation method, to thereby confirm that the correct antigen peptide has been produced.

In the case where a relatively low molecular peptide, such as a peptide having an amino acid sequence of SEQ ID NO: 1, 2, or 3, is used as an antigen, the peptide is preferably bound to and carried by a carrier such as hemocyanin, ovoalbumin, or γ-globulin.

Depending on whether the antibody is monoclonal or polyclonal, one of the following schemes is selected for producing the antibody.

A monoclonal antibody may be produced by use of the aforementioned antigen peptide in accordance with the method described by Kohler and Milstein (Nature 256, 495-497 (1975)).

For example, an antigen peptide is administered to an animal for immunization; e.g., a mouse, a rat, a guinea pig, a rabbit, a goat, sheep, a horse, a pig, a dog, a cat, or a chicken, intraperitoneally, subcutaneously, through a footpad, or by any other suitable means. Among the listed animals, a mouse is preferred. In other words, the antibody used in the present invention is preferably derived from a mouse.

From the thus-immunized animal, spleen cells, lymphocytes, peripheral blood, or a similar sample is collected, followed by fusion with a tumor cell line; i.e., myeloma cells, to thereby produce a hybridoma.

The obtained hybridoma is subjected to continuous culturing for proliferation, then screened for a hybridoma strain which consistently produces an antibody capable of binding specifically to the antigen.

The thus-screened hybridoma strain is cultured in a suitable medium, to thereby yield a monoclonal antibody in the medium. Alternatively, the hybridoma cells may be cultured in a living body; for example, the hybridoma cells can be cultured in the abdominal cavity of a mouse, followed by isolation from the ascites, thus enabling mass production of monoclonal antibody. The obtained monoclonal antibody may be purified through a conventional purification method for an antibody.

A polyclonal antibody may be produced by use of the aforementioned antigen peptide in the following manner.

In an analogous manner to that employed in the case of producing a monoclonal antibody, an antigen peptide is administered to an animal for immunization. Here, a rabbit is a preferred animal for immunization.

When immunization is performed, combined use of an adjuvant is preferred for activation of antibody-producing cells. If the animal is boosted by a routine method 2 to 3 weeks after the initial immunization, antiserum with a high titer can be obtained. About one week after the final immunization, blood is collected and serum is separated therefrom. The thus-collected serum can be treated with heat, to thereby deactivate complements, and subsequently subjected to a conventional purification procedure, to thereby purify immunoglobulin fractions.

Persons with ordinary skill in the art would easily determine, by use of a conventional method employing an antigen peptide or any other substance which can serve as an antigen peptide, whether or not the produced antibody can bind to the antigen peptide, or can bind to the antigen peptide specifically.

Preferably, the immunoglobulin subclass of the thus-obtained antibody is IgG1. Antibodies whose immunoglobulin subclass is IgG1 can be obtained through, for example, screening employing an anti-IgG1 antibody.

A particularly preferred example of such an antibody is an antibody capable of binding, specifically, to a peptide having an amino acid sequence of SEQ ID NO: 2, which, preferably, is a monoclonal antibody. Also, preferably, the immunoglobulin subclass of this antibody is, as has already been mentioned, IgG1.

Another preferred example of the antibody of the present invention is an antibody capable of binding, specifically, to a peptide having an amino acid sequence of SEQ ID NO: 3, which, preferably, is a polyclonal antibody.

According to the present invention, the antibody may be in a labeled form, or in a non-labeled form but capable of being labeled with a labeling substance. No particular limitations are imposed on the labeling substance, so long as it can be used in an ordinary protein labeling procedure. Examples of employable labeling substances include an enzyme (e.g., peroxidase or alkaline phosphatase), a radioisotope (e.g., ¹²⁵I or ¹³¹I), a fluorescent dye (e.g., Alexa Fluor (registered trademark) 488 or fluorescein isothiocyanate (FITC)), chemiluminescent substance (e.g., luminol), a hapten (e.g., dinitrofluorobenzene), and one of the substances that constitute a specific binding pair (e.g., biotin or an avidin, such as streptoavidin).

The method for labeling the antibody with a labeling substance may be appropriately selected from among known methods suited for the labeling substance.

Preferably, measurement making use of antigen-antibody reaction is performed by use of a solid phase. In the present invention, no particular limitations are imposed on the solid phase, so long as it permits a protein (e.g., CAP 18 or an antibody) to be immobilized thereon and is insoluble to water, a biological sample, or a reaction mixture related to the measurement. Examples of the solid phase include a plate (e.g., wells of a microplate), a tube, a bead, a membrane, a gel, and a finely divided solid carrier (e.g., gelatin particles, kaolin particles, or synthetic polymer particles such as latex particles). In consideration of accurate quantitation and convenience of use, a microplate is preferred.

The material that forms the solid phase may be, for example, polystyrene, polypropyrene, polyvinyl chloride, nitrocellulose, nylon, polyacrylamide, Teflon (registered trademark), polyallomer, polyethylene, glass, or agarose. Use of a plate made of polystyrene is preferred.

The measurement making use of antigen-antibody reaction employing a solid phase is preferably performed through a method including at least the following steps (a) to (c) (Method 1):

(a) a step of bringing a sample into contact with a solid phase, to thereby immobilize onto the solid phase CAP 18 contained in the sample;

(b) a step of causing the immobilized CAP 18 obtained in step (a) to be bound to “an antibody capable of binding to a peptide having an amino acid sequence of SEQ ID NO: 1,” to thereby form a complex of the two components; and

(c) a step of detecting the complex formed in step (b).

In step (a), no particular limitations are imposed on the method for bringing a sample into contact with a solid phase, so long as CAP 18 molecules contained in the sample contact a surface of the solid phase. For example, the sample may be added to the solid phase, so as to establish contact therebetween, or alternatively, the solid phase may be added to the sample for achieving contact therebetween. Further alternatively, the sample and the solid phase may be simultaneously added to a separate container. However, manner of achieving contact is not limited to only these method, and may be adequately determined by one skilled in the art, in accordance with the shape, material, etc. of the solid phase. Through the mentioned contact, CAP 18 contained in the sample is bound to the solid phase. In order to achieve ensured binding, preferably, incubation is performed at 4 to 37° C. for 1 hour to overnight.

After the solid phase has been brought into contact with the sample, solid-liquid phase separation is performed. Preferably, nonspecifically adsorbed substances and unbound substances are removed by washing the surfaces of the solid phase with. a washing solution in accordance with needs.

A preferred example of the washing solution is a buffer (such as phosphate buffer, PBS, or a Tris-HCl buffer) to which a nonionic surfactant such as Tween has been added.

In step (b), in order to cause the immobilized CAP 18—immobilized on the solid phase—to be bound to “an antibody capable of binding to a peptide having an amino acid sequence of SEQ ID NO: 1,” the immobilized CAP 18 and the antibody are brought into contact with each other under conditions that are conventionally employed for inducing an antigen-antibody reaction. In order to obtain sufficient binding between CAP 18 and the antibody, the two are preferably incubated at 4 to 37° C. (more preferably 20 to 37° C.) for 1 to 4 hours or thereabouts, after establishment of contact. Through this procedure, a complex between the two is formed.

Thereafter, the liquid and solid phases are separated from each other, and the surfaces of the solid phase are washed with a washing solution in accordance with needs. The employable washing solution can be determined by analogy to the above.

In step (c), no particular limitations are imposed on the method of detecting the complex formed in step (b). For example, when the antibody has been labeled with a labeling substance, the complex can be detected by detecting the labeling substance attached to the antibody which forms the complex. In order to detect a labeling substance, any suitable conventionally known detection method may be employed in accordance with the identity of the labeling substance. Descriptions related to other matters are omitted, as those provided hereinabove can be applied similarly.

Alternatively, the measurement making use of antigen-antibody reaction employing a solid phase is preferably performed through a method including at least the following steps (a)′ and (b)′ (Method 2):

(a)′: a step of bringing into mutual contact the following three components; i.e., a solid phase to which a first antibody capable of binding to a peptide having an amino acid sequence of SEQ ID NO: 1 has been immobilized, a sample, and a second antibody capable of binding to a peptide having an amino acid sequence of SEQ ID NO: 1, to thereby form a sandwich-like complex formed of “first antibody immobilized onto a solid phase—CAP 18—second antibody”; and

(b)′ a step of detecting the sandwich-like complex formed in step (a)′.

In step (a)′, the “solid phase to which a first antibody capable of binding to a peptide having an amino acid sequence of SEQ ID NO: 1 has been immobilized” can be prepared by causing the first antibody to be immobilized onto a solid phase through any appropriate means.

For devising such a means, there may be applied a conventional method for preparing an immobilized enzyme, such as physical adsorption, covalent bonding, or entrapment (see “Immobilized Enzyme,” 1975, published by Kodansha, pp. 9-75).

Of these methods, physical absorption is preferred, because it requires only simple and convenient operation and is widely employed in this technical field.

The surface of the solid phase which has been brought into contact with the first antibody for achieving binding therebetween may have some portions left unbound to the first antibody, and therefore, when CAP 18 present in a sample is bound to such an unbound region in a non-specific manner, accurate measurement cannot be achieved. Thus, preferably, before the sample is brought into contact with the solid phase, a blocking substance is added, so as to cover the portions remaining unbound to the first antibody. Examples of the blocking substance include serum, serum albumin, casein, skim milk, gelatin, and pluronic. Alternatively, a commercially available blocking agent may be employed.

No particular limitations are imposed on the first and the second antibody used in Method 2, so long as they are antibodies capable of binding to a peptide having an amino acid sequence of SEQ ID NO: 1. Preferably, the first antibody is an antibody capable of binding to a peptide having an amino acid sequence of SEQ ID NO: 3, and the second antibody is an antibody capable of binding to a peptide having an amino acid sequence of SEQ ID NO: 2.

In step (a)′, the three components; i.e., a solid phase to which a first antibody capable of binding to a peptide having an amino acid sequence of SEQ ID NO: 1 has been immobilized, a sample, and a second antibody capable of binding to a peptide having an amino acid sequence of SEQ ID NO: 1, may be brought into contact with one another simultaneously. In an alternative procedure, firstly the former two components are brought into contact with each other, and then the third component is added for contact, or firstly the latter two components are brought into contact with each other, and then the first component is added for contact. Preferably, the former two components are first brought into contact with each other, and then the third component is added.

No particular limitations are imposed on the method for achieving contact between components, so long as the molecules of the first antibody that have been immobilized onto the solid phase, CAP 18 molecules contained in the sample, and the molecules of the second antibody are allowed to come into contact with one another, and therefore, there can be employed any contacting method performed under conventional conditions for inducing antigen-antibody reaction. In order to obtain sufficient binding between CAP 18 and the antibody, after the two components are brought into contact with each other, they are preferably incubated at 4 to 37° C. (more preferably 20 to 37° C.) for 1 to 4 hours or thereabouts.

Through this procedure, a sandwich-like complex formed of “first antibody immobilized onto a solid phase—CAP 18—second antibody” is obtained.

Subsequently, the solid and liquid phases are separated from each other, and the surfaces of the solid phase are washed with a washing solution in accordance with needs. The washing solution which may be used is analogous to those described hereinabove.

In step (b)′, no particular limitations are imposed on the method for detecting the sandwich-like complex. For example, when the second antibody is labeled with a labeling substance, the sandwich-like complex can be detected through detection of the labeling substance attached to the second antibody which forms the complex. In order to detect a labeling substance, any suitable conventionally known detection method may be employed in accordance with the identity of the labeling substance. Descriptions related to other matters are omitted, as those provided hereinabove can be similarly applied.

When measurement is performed as described above, the amount of CAP 18 contained in a biological sample can be obtained. The thus-obtained measurement may be used directly for establishing correlation with CF. Alternatively, the measurement may be compared with the amount of CAP 18 contained in a control sample, and the results of comparison used for establishing correlation with CF. The control sample may be suitably selected in accordance with the purpose of assessment.

For example, when the purpose of assessment is diagnosing CF, or determination of the presence or absence of risk of CF or assessment of the level of the risk, the control sample may be a biological sample collected from a healthy individual (not suffering from CF), or a biological sample collected from an individual suffering from CF. Alternatively, when the purpose is assessment of severity and/or acuteness, or assessment regarding progress of disease, the control sample may be biological samples collected from a specific individual at certain time intervals.

According to the present invention, the “assessment” is preferably selected from among diagnosis; determination as to the presence or absence of risk, or assessment of the level of the risk; assessment of severity and/or acuteness; and assessment regarding progress of disease.

The present inventors have found that the level of CAP 18 contained in a biological sample collected from an individual suffering from CF is significantly high. Therefore, on the basis of this finding, CF can be correlated with the amount of CAP 18 determined by the measurement or with the results obtained from comparing this value with the measurement value determined in a control sample.

Specifically, the following correlation can be established. When the CAP 18 level (amount of CAP 18) of a biological sample measures higher than the corresponding value as measured for a healthy (not suffering from CF) individual, the correlation may be “CF is confirmed,” “high possibility of CF being confirmed,” or “high risk of CF.”

Alternatively, when the CAP 18 level (amount of CAP 18) of a biological sample measures almost equal to or lower than the corresponding value as measured for a healthy individual, the correlation may be “not suffering from CF,” “low possibility of CF being confirmed,” or “low risk of CF.”

Also, the magnitude of the subtraction difference between CAP 18 level (amount of CAP 18) of a biological sample and a CAP 18 level (amount of CAP 18) determined for a healthy individual can be correlated with the severity of CF.

Moreover, from a certain, specific individual, biological samples are collected at predetermined intervals for measurement of the CAP 18 level, and when tendency of increasing CAP 18 level is revealed, such a tendency can be correlated with “CF is under progress” or “high possibility of CF being under progress.” Conversely, when decreasing tendency of CAP 18 level is revealed, such a tendency can be correlated to “CF is under amelioration” or “high possibility of CF being under amelioration. Also, when no change is observed in the level of CAP 18, this finding can be correlated to “stable condition of CF in terms of aggravation (or amelioration)” or “high possibility of CF being in stable condition in terms of aggravation (or amelioration).

Depending on the type, etc. of a biological sample, the CAP 18 level may decrease when the donor of the sample suffers from CF. In such a case, correlation can be established in a manner converse to that described above.

<2> The Present Kit

Kit 1 of the present invention is directed to a kit for assessment of CF, containing at least the following components (A) and (B):

(A) a solid phase, and

(B) an antibody capable of binding to a peptide having an amino acid sequence of SEQ ID NO: 1.

Preferably, the antibody (B) is a labeled antibody which has been labeled, or can be labeled, with a labeling substance.

Previous descriptions provided hereinabove have already addressed the solid phase, the antibody capable of binding to a peptide having an amino acid sequence of SEQ ID NO: 1, the labeling substance, the CAP 18 to be measured, and assessment of CF, and therefore, repeated descriptions are omitted, in view that the same are applied thereto. Kit 1 may be used in accordance with Method 1 of the present invention.

Kit 2 of the present invention is directed to a kit for assessment of CF, containing at least the following components (A)′ and (B)′:

(A)′: a solid phase to which a first antibody capable of binding to a peptide having an amino acid sequence of SEQ ID NO: 1 has been immobilized, and

(B)′: a second antibody capable of binding to a peptide having an amino acid sequence of SEQ ID NO: 1.

Preferably, the second antibody (B)′ is a labeled antibody which has been labeled, or can be labeled, with a labeling substance.

Previous descriptions provided hereinabove have already addressed the first antibody, the second antibody, the solid phase to which the first antibody has been immobilized, the labeling substance, the CAP 18 to be measured, and assessment of CF, and therefore, repeated descriptions are omitted, in view that the same are applied thereto. Kit 2 may be used in accordance with Method 2 of the present invention.

No particular limitations are imposed on the present kit, so long as it includes at least the listed components. In addition to the essential components, the kit may contain, as a component thereof, a standard CAP 18 sample having a known concentration—which may serve as a standard for drawing a calibration curve or establishing a correlation formula—or a reagent for detecting a labeling substance. Furthermore, in addition to these components, the kit may contain other optional components, including a blocking substance, a washing solution, a solution for diluting a biological sample, and an enzymatic reaction stopping solution. Moreover, the present kit may include a positive control (QC control) for maintaining the level of assay performance consistent among a plurality of measurement batches.

### EXAMPLES

The present invention will next be described in more detail by way of examples, which should not be construed as limiting the invention thereto.

Production Examples

(1) Production of Monoclonal Antibody

A peptide having an amino acid sequence of SEQ ID NO: 1 (FR KSKEK IGKEF KRIVQ RIKDF LRNLV, hereinafter referred to as the “27-amino-acid peptide”) was synthesized and conjugated to hemocyanin (keyhold lympet hemocyanin). The product was intraperitoneally administered to a mouse (Balb/c) for immunization. A complete adjuvant was employed for the first immunization, and thereafter an incomplete adjuvant was employed. Spleen cells were collected from the thus-immunized mouse, and fused with mouse myeloma cells (cell line: P3-X63-Ag8.653) by use of polyethylene glycol 4000 (PEG4000), to thereby produce hybridomas.

The hybridomas were subjected to continuous culturing for proliferation, then screened for a hybridoma strain which consistently produces an antibody capable of binding specifically to the 27-amino-acid peptide.

The isolated hybridoma strain was cultured in a serum-free medium (trade name: CD hybridoma, product of Invitrogen) by use of a hollow-fiber bioreactor. The culture supernatant was isolated and dialyzed against PBS, to thereby obtain a monoclonal antibody (Toyo 6E3). The immunoglobulin subclass of the antibody was identified as IgG1.

(2) Production of Polyclonal Antibody

A recombinant CAP 18 (derived from human) was subcutaneously administered to a rabbit for immunization. A complete adjuvant was employed for the first immunization, and thereafter an incomplete adjuvant was employed. Two to three weeks after the first immunization, the rabbit was boosted through a conventional method. About one week after the final immunization, blood was collected from the rabbit, and serum was separated therefrom. The serum was subjected to heat treatment, to thereby inactivate the complements, followed by treatment with 33% saturated ammonium sulfate for causing precipitation, to thereby prepare a polyclonal antibody.

### Example 1

### Assessment of CF

(1) Method for Measurement of CAP 18

The polyclonal antibody (first antibody) prepared in Production Example (2) was dissolved in Tris buffer (pH 7.4), and the solution was added to wells of a polystyrene microtiter plate and incubated at 22° C. or thereabouts for one hour, to thereby cause physical adsorption of the polyclonal antibody onto the plate. The plate was washed with Tris buffer, after which a Tris buffer supplemented with 1% FBS was added to the wells of the plate for blocking. Separately, a blood sample was collected from a living organism, and the sample was centrifuged at 2,000 rpm for 10 minutes, to thereby obtain a supernatant (biological sample). An aliquot of the supernatant was added to each well of the plate, followed by incubation at 22° C. for three hours. After completion of incubation, the plate was washed with Tris buffer.

Subsequently, the monoclonal antibody (Toyo 6E3) prepared in Production Example (1) was added to the wells of the plate, followed by incubation at 22° C. for two hours. After completion of incubation, the plate was washed with Tris buffer.

Subsequently, goat anti-mouse IgG antibody which had been labeled with HRP was added to the wells of the plate, and reaction was caused to proceed in a manner similar to that described above. A TMB chromogen was added for allowing color to develop. Absorbance at 450 nm was measured. The amount of CAP 18 was calculated on the basis of the absorbance and a calibration curve which had been prepared by use of recombinant CAP 18 (derived from human).

(2) Evaluation of CF by use of BALF

BALF samples obtained from patients suffering CF and healthy humans were employed as biological samples, and the amount of CAP 18 contained in each sample was measured through the measurement method described in (1) above. The results (“mean±SD”) are shown below.

BALF samples from CF patients (n=23) 189.7±18.7 (μg/mL)

BALF samples from healthy humans (n=12) 120.7±24.7 (μg/mL)

p=0.036 (unpaired 2-tail t test)

These results indicate that the CAP 18 levels of BALF samples determined for the patients suffering CF are significantly higher than those determined for healthy humans. Accordingly, when the CAP 18 level of a BALF sample of a certain individual is high, the measurement can be correlated with “CF is confirmed” or “high possibility of CF being confirmed.”

(3) Evaluation of CF by use of Expectoration

Expectoration samples obtained from patients suffering CF were employed as biological samples, and the amount of CAP 18 contained in each sample was measured through the measurement method described in (1) above. The results (“mean±SD”) are shown below.

Expectoration samples from CF patients (n=30) 177.4 ±14.7 (μg/mL)

These results reveal that the CAP 18 levels of expectoration samples from the patients suffering CF are as high as those of BALF samples. Accordingly, when the CAP 18 level of an expectoration sample of a certain individual is high, the measurement can be correlated with “CF is confirmed” or “high possibility of CF being confirmed.”

### Example 2

### Preparation of Kit 1

A Kit 1 of the present invention containing the following components was prepared:
1. polystyrene microtiter plate: 1 plate;
2. monoclonal antibody (Toyo 6E3) (first antibody) prepared in Production Example (1): 1 vial;
3. goat anti-mouse IgG antibody (second antibody) labeled with HRP: 1 vial;
4. TMB solution: 1 vial;
5. reaction stopping solution (1N HCl): 1 vial;
6. washing solution (PBS containing 0.05% Tween20);
7. solution for diluting biological sample (PBS(−) containing 1% BSA);
8. CAP 18 standard solution: 1 set; and
9. manual describing method for assessment of CF, etc.

### Example 3

### Preparation of Kit 2

A Kit 2 of the present invention containing the following components was prepared:
1. polystyrene microtiter plate to which polyclonal antibody prepared in Production Example (2) has been immobilized: 1 plate;
2. monoclonal antibody (Toyo 6E3) (first antibody) prepared in Production Example (1): 1 vial;
3. goat anti-mouse IgG antibody (second antibody) labeled with HRP: 1 vial;
4. TMB solution: 1 vial;
5. reaction stopping solution (1N HCl): 1 vial;
6. washing solution (PBS containing 0.05% Tween20);
7. solution for diluting biological sample (PBS(−) containing 1% BSA);
8. CAP 18 standard solution: 1 set; and
9. manual describing method for assessment of CF, etc.

As described hereinabove, the method of the present invention is very useful for assessment of CF, because it provides very accurate, highly sensitive, convenient, rapid, and inexpensive assessment. Moreover, the present method may employ a biological sample collected in a noninvasive manner, such as an expectoration, thereby greatly reducing the burden imposed on the patient and making the method very practical. The kit of the present invention is also very useful, because it ensures quicker and more convenient performance of the method of the invention.

In addition, the present invention finds remarkable utility in a variety of applications, including identification of the status of CF, determination of therapeutic regimen, confirmation of therapeutic effect, observation of the course of therapy, assessment related to development of drugs, etc.

1. A method for assessment of cystic lung fibrosis, comprising measuring the level of CAP 18 in a biological sample, and correlating the measurement with cystic lung fibrosis.

2. A method for assessment of cystic lung fibrosis, comprising the following steps (1) to (3):
(1) a step of measuring the level of CAP 18 contained in a biological sample collected from an individual;
(2) a step of comparing the CAP 18 level determined in step (1) with the level of CAP 18 in a control sample; and
(3) a step of correlating the result from step (2) with cystic lung fibrosis.

3. The method according to claim 1, wherein the biological sample is expectoration or bronchoalveolar lavage fluid (BALF).

4. The method according to claim 1, wherein the level of CAP 18 is measured through antigen-antibody reaction.

5. The method according to claim 4, wherein the measurement through antigen-antibody reaction employs an antibody capable of binding to a peptide having an amino acid sequence of SEQ ID NO: 1.

6. The method according to claim 4, wherein the measurement through antigen-antibody reaction employs a solid phase.

7. The method according to claim 6, wherein the measurement through antigen-antibody reaction employing a solid phase is performed through a method comprising the following steps (a) to (c):
(a) a step of bringing a sample into contact with a solid phase, to thereby immobilize onto the solid phase CAP 18 contained in the sample;
(b) a step of causing the immobilized CAP 18 obtained in step (a) to be bound to “an antibody capable of binding to a peptide having an amino acid sequence of SEQ ID NO: 1,” to thereby form a complex of the two components; and
(c) a step of detecting the complex formed in step (b).

8. The method according to claim 6, wherein the measurement through antigen-antibody reaction employing a solid phase is performed through a method comprising the following steps (a)′ and (b)′:
(a)′: a step of bringing into mutual contact the following three components; i.e., a solid phase to which a first antibody capable of binding to a peptide having an amino acid sequence of SEQ ID NO: 1 has been immobilized, a sample, and a second antibody capable of binding to a peptide having an amino acid sequence of SEQ ID NO: 1, to thereby form a sandwich-like complex formed of “first antibody immobilized onto a solid phase—CAP 18—second antibody”; and
(b)′ a step of detecting the sandwich-like complex formed in step (a)′.

9. The method according to claim 1, wherein the assessment is selected from the group consisting of diagnosis; determination of the presence or absence of risk, or assessment of the level of the risk; assessment of severity and/or acuteness; and assessment regarding progress of disease.

10. The method according to claim 2, wherein the biological sample is expectoration or bronchoalveolar lavage fluid (BALF).

11. The method according to claim 2, wherein the level of CAP 18 is measured through antigen-antibody reaction.

12. The method according to claim 11, wherein the measurement through antigen-antibody reaction employs an antibody capable of binding to a peptide having an amino acid sequence of SEQ ID NO: 1.

13. The method according to claim 11, wherein the measurement through antigen-antibody reaction employs a solid phase.

14. The method according to claim 13, wherein the measurement through antigen-antibody reaction employing a solid phase is performed through a method comprising the following steps (a) to (c):
(a) a step of bringing a sample into contact with a solid phase, to thereby immobilize onto the solid phase CAP 18 contained in the sample;
(b) a step of causing the immobilized CAP 18 obtained in step (a) to be bound to “an antibody capable of binding to a peptide having an amino acid sequence of SEQ ID NO: 1,” to thereby form a complex of the two components; and
(c) a step of detecting the complex formed in step (b).

15. The method according to claim 13, wherein the measurement through antigen-antibody reaction employing a solid phase is performed through a method comprising the following steps (a)′ and (b)′:
(a)′: a step of bringing into mutual contact the following three components; i.e., a solid phase to which a first antibody capable of binding to a peptide having an amino acid sequence of SEQ ID NO: 1 has been immobilized, a sample, and a second antibody capable of binding to a peptide having an amino acid sequence of SEQ ID NO: 1, to thereby form a sandwich-like complex formed of “first antibody immobilized onto a solid phase—CAP 18—second antibody”; and
(b)′ a step of detecting the sandwich-like complex formed in step (a)′.

16. The method according to claim 2, wherein the assessment is selected from the group consisting of diagnosis; determination of the presence or absence of risk, or assessment of the level of the risk; assessment of severity and/or acuteness; and assessment regarding progress of disease.

17. A kit for assessment of cystic lung fibrosis, comprising the following components (A) and (B):
(A) a solid phase, and
(B) an antibody capable of binding to a peptide having an amino acid sequence of SEQ ID NO: 1.

18. A kit for assessment of cystic lung fibrosis, comprising the following components (A)′ and (B)′:
(A)′: a solid phase to which a first antibody capable of binding to a peptide having an amino acid sequence of SEQ ID NO: 1 has been immobilized, and
(B)′: a second antibody capable of binding to a peptide having an amino acid sequence of SEQ ID NO: 1.


SEQUENCE LISTING (4 sequences)

SEQ ID NO: 1 - protein, 27 aa, Artificial Sequence
  Antigen sequence
FRKSKEKIGKEFKRIVQRIKDFLRNLV

SEQ ID NO: 2 - protein, 18 aa, Artificial Sequence
  Antigen sequence
KEFKRIVQRIKDFLRNLV

SEQ ID NO: 3 - protein, 9 aa, Artificial Sequence
  Antigen sequence
FRKSKEKIG

SEQ ID NO: 4 - protein, 170 aa, Homo sapiens
MKTQRNGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES